(19) United States
(12) Patent Application Publication
(10) Pub. No.: US 2003/0104451 A1
(43) Pub. Date: Jun. 5, 2003

(54) Arrays using polymerized monomolecular films and methods for using and manufacturing the same

(76) Inventors: Susan K. Hobbs, Fremont, CA (US); Mark D. Bednarski, Los Altos, CA (US); Yi-Shan Yang, Saratoga, CA (US); Samira Guccione, Hillsborough, CA (US); Gongyi Shi, Dublin, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/285,810

(22) Filed: Oct. 31, 2002

Related U.S. Application Data
(60) Provisional application No. 60/334,701, filed on Oct. 31, 2001.

(51) Int. Cl. C12Q 1/68; G01N 33/53; C12M 1/34

(52) U.S. Cl. 435/6; 435/7.1; 435/287.2

(57) ABSTRACT

Devices and methods of use and manufacture for the identification and characterization of analytes, e.g. proteins, are provided. The subject devices are characterized by having a substrate with a polymerized monomolecular film over at least a portion of the substrate, the monomolecular film having at least one ligand or specific binding pair member associated therewith. Preferably the monomolecular film is stable to the laser intensities employed in MALDI-MS. In certain embodiments, the ligands are biotin, integrin antagonists, antibodies and antigens. In using the subject devices, a subject device is contacted with a sample. If present in the sample, a member of the binding pair of interest binds to its complementary ligand and, once bound, can be analyze by mass spectroscopy techniques. Also provided are kits, which include the subject devices.

### BACKGROUND OF THE INVENTION

A current emphasis in proteomics is the development of materials for use in immobilized microarrays for protein detection similar to that of gene expression analysis. Currently, materials for microarray development have focused on arraying biomolecules in a manner that retains their biological activity. Antibodies, small molecules, peptide ligands, purified recombinant proteins, and whole tissue preparations have been non-specifically arrayed on different materials. A more difficult problem is to design materials that can capture specific proteins and limit nonspecific binding to the surface. So far, the investigation of small molecule or peptide arrays have been limited to model systems that contain only purified proteins binding to their respective ligands displayed on the material surface. In addition, the captured biomolecule is often present at low concentrations and must be accurately detected and quantified. No materials have demonstrated the capability to both reproducibly capture specific proteins from complex protein mixtures and be amenable for rapid mass spectral analysis.

For example, one current approach to protein identification involves isolating protein using two dimensional polyacrylamide gel electrophoresis (2D-PAGE) followed by enzyme degradation of the isolated protein spots on the gel and the preparation of peptide maps and bioinformatic searches. To perform 2D-PAGE, at least hundreds of femtomoles to low picomoles of isolated protein must be present on a single gel spot in order to identify the protein and/or characterize it. Thus, significant drawbacks to 2D-PAGE include, but are not limited to, time consuming processes, large sample requirements, time consuming sample preparation, low protein solubilization, separation of low abundance proteins from complex mixtures difficulties, high sample loads needed to evaluate co-translational and post-translational modifications and difficulties when used with highly basic proteins and very large (greater than 150 kDa) or very small (less than 10 kDa) proteins. In addition to 2D-PAGE, methods such as 2D-liquid chromatography and capillary zone electrophoresis have been employed. However, these also suffer from similar problems, i.e., lengthy processes, etc.

Recently, chemical modification of surfaces by organic molecules or films has been successfully used to identify and/or characterize analytes such a proteins in a sample. Such chemically modified surfaces are commonly known as arrays (also known as microarrays). These arrays, in which a plurality of ligands or members of a specific binding pair are deposited onto a solid support surface in the form of an “array” or pattern, find use in a variety of applications, including, but not limited to, proteomic identification and analysis, gene expression analysis, drug screening, nucleic acid sequencing, mutation analysis, and the like.

For example, the techniques of molecular self-assembly (Science (261), 1993) and Langmuir-Blodgett deposition (LB) (Introduction to Ultrathin Organic Films, A Ullman 1991; Langmuir-Blodgett Films, G. Roberts 1990) have been used for coating surfaces with a well-defined, quasi-two-dimensional array of molecules. Currently, efforts in the area of arrays have been focused on the development of methods that utilize protein function and protein-protein interactions or methods for capturing a known protein in cell lysate using antibody arrays. However, these arrays are not without problems. One significant problem associated with arrays involves the difficulty, cost and time required for the high throughput production and purification of the surface-bound proteins.

As such, there is continued interest in the development of array based devices and methods of use thereof for analyte, e.g., protein, identification and characterization. Of particular interest would be the development of such devices that are easy and inexpensive to use and manufacture, are stable to mass spectroscopy analysis and in particular matrix-associated laser desorption/ionization mass spectroscopy (MALDI-MS), are easily customizable, have high protein-binding specificity and have low non-specific protein adhesion.

A potential solution to many of these problems is to chemically modify surfaces with organic monomolecular films such as self-assembled monolayers (SAMs) (Mrksisch & Whitesides (1995) Trends in Biotechnology 13, 228). These films, which are formed on a platform surface by the spontaneous adsorption of molecules from solution, have important properties that can be utilized for proteomic analysis using MALDI-MS. The films are chemically and physically flexible, thermodynamically and kinetically stable, and provide homogeneous surface coverage (Mitchell (2002) Nature Biotechnology 20:225-229). Polymerized diacetylene thin films (PDTFs) are unique SAMs with potential applications in microarrays because the polymerized films are stable to laser desorption and can appropriately display ligands for subsequent protein capture with low nonspecific protein binding (Charych et al. (1993) Science 261:585-588). PDTFs can be arranged on a variety of platform surfaces and have been characterized using X-ray photoelectron spectroscopy (XPS), ellipsometry, fluorescent microscopy, contact angle measurement, atomic force microscopy, and scanning tunneling microscopy (Wilson et al. (1992) Langmuir 8, 2361-2364); Wilson et al. (1992) Langmuir 8, 2588-2590). These studies demonstrated that PDTFs produce monolayers with the ligand functional groups directed towards the ambient surface, a property that is critical for proper binding of proteins.

### SUMMARY OF THE INVENTION

Devices and methods of use and manufacture for the identification and characterization of analytes, e.g., proteins, are provided. A substrate is provided with a functionalized polymerized monomolecular film (PTF) over at least a portion of the substrate, the polymerized monomolecular film having at least one ligand or specific binding pair member associated therewith. Functionalized polymerized thin film surfaces are constructed with self-assembling monomers containing a ligand lipid monomer. Functionalized PTFs can be easily manufactured for customized microarrays, demonstrate high protein specificity, low non-specific protein adsorption, and the resulting microarrays constructed from these materials are compatible with several different MS protein analysis platforms.

In one embodiment of the invention, the substrate comprises self-assembled polymerized diacetylene thin films (PDTFs); and specific binding member-functionalized lipids, which films are stable to the laser intensities employed in MALDI-MS and display ligands at the interface-to capture proteins for MS detection.

### BRIEF DESCRIPTION OF THE DRAWINGS

FIGS. 1A-1C. Materials for immobilized ligand display (MILD) array. A) Method for constructing PDTFs. A modified hydrophobic aluminum oxide surface was used as a solid support for PDTF formation. An aqueous phase is applied to the base surface followed by an organic phase containing the diacetylene monomeric compounds. Polymerization is achieved through ultraviolet irradiation (254 nm for approximately 15 minutes). The materials are then cured at room temperature for 24 hours. B) Chemical structures of PDTFs. Schematic representation of biotin, hydroxyl, and integrin antagonist terminated head groups. These ligands are immobilized and displayed within the PDTF backbone. C) Fluorescent properties of PDTFs. Fluorescent micrograph obtained at 10× magnification using FITC filters (PDTFs exhibit an excitation at 480 nm and emission at 525 nm)¹⁶. Note the complete coverage and homogeneity of the film within the 2 mm spot size using the above arraying method.

FIG. 2. Streptavidin protein capture and detection with MILD-MALDI MS. The peak streptavidin area was plotted against percentage of biotin contained in the PDTF. The mean and standard deviation of triplicate runs are shown. The PDTFs are EAPDA-PDTF, 0.1% (0.025 pmol/layer) Biotin-PDTF, 1% (0.25 pmol/layer) Biotin-PDTF, and 10% (2.5 pmol/layer) Biotin-PDTF. All surfaces were incubated with the same amount of streptavidin (2.75 pmol).

FIGS. 3A and 3B. Sensitivity with MILD-MALDI MS. A The peak streptavidin area was plotted against percentage of streptavidin contained in 1 μg/ml total protein applied to the chip surface. The mean and standard deviation of triplicate runs are shown. All surfaces contain 2.5 pmol/monolayer biotin. B Representative MILD-MALDI MS spectra. Peak of streptavidin, as indicated by arrows, is detectable at 0.2% of total protein in solution in this example. Specific protein binding is demonstrated by the relative absence of other protein peaks.

FIGS. 4A-4B. Competitive inhibition. A The peak streptavidin area was plotted against the amount of biotin contained in the incubation solution prior to application to the MILD-PDTF surface. The mean and standard deviation of triplicate runs are shown. Streptavidin (0.01 μM; 50 fmole) was incubated with 0.1 μM, 1 μM, or 10 μM of free biotin for 30 minutes prior to incubation on the MILD-PDTF (0.25 pmol/monolayer biotin), surface. B Representative MILD-MALDI-MS spectra. Peak of streptavidin, indicated by arrows decreases with increasing amount of biotin in solution.

FIG. 5. Integrin αᵥβ₃ protein capture and detection with MILD-MALDI-MS. A purified αᵥβ₃ integrin (20 fmol) in PBS on 30% integrin antagonist-PDTF, B purified αᵥβ₃ (20 fmol) in cellular lysate (1 mg/ml) on 30% integrin antagonist-PDTF, C purified αᵥβ₃ (20 fmol) in cellular lysate (1 mg/ml) on EAPDA-PDTF. Thin arrows indicate the expected mass peak of the integrin subunit αᵥ; thick arrows indicate peak of the β₃ subunit

### DETAILED DESCRIPTION OF THE EMBODIMENTS

Devices and methods of use and manufacture for the identification and characterization of analytes, e.g., proteins, are provided. The subject devices are characterized by having a substrate with a polymerized monomolecular film over at least a portion of the substrate, the monomolecular film having at least one ligand or specific binding pair member associated therewith. Preferably the monomolecular film is stable to the laser intensities employed in MALDI-MS. In using the subject devices to identify and/or characterize one or more analytes, a subject device having a substrate with a polymerized monomolecular film over at least a portion of the substrate, wherein the polymerized monomolecular film comprises at least one ligand or binding pair member associated therewith, is contacted with a sample. If the analyte of interest is present in the sample, it will bind to the complementary member of the binding pair and, once bound, can be analyzed by mass spectroscopy, such as matrix-associated laser desorption/ionization mass spectroscopy (MALDI-MS). Also provided are kits, which include the subject devices.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a device” includes a plurality of such devices and reference to “the array” includes reference to one or more arrays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Polymerized Monomolecular Film. As used herein, the term “films” refers to a material deposited or used in a thin section or in a layer form, where the film is a single molecule thick. The thin films of the invention are comprised of two components, a ligand (or specific binding moiety) containing amphipathic molecules, and a self-assembling monomer. As used herein, the terms “self-assembling monomers” and “lipid monomers” refer to molecules that spontaneously associate to form molecular assemblies. In one sense, this can refer to surfactant molecules that associate to form surfactant molecular assemblies. The term “self-assembling monomers” includes single molecules, e.g. a single lipid molecule) and small molecular assemblies, e.g. polymerized lipids, whereby the individual small molecular assemblies can be further aggregated, e.g. assembled and polymerized, into larger molecular assemblies. Generally the ratio in the film of ligand containing molecules to self-assembling monomers will be at least about 0.1, about 1, about 10, about 15, or about 20 mole percent.

The polymerized monomolecular film comprises self-assembling monomers having a crosslinking functional group, e.g. diacetylene, olefins, acetylenes, nitriles, alkyl styrenes, esters, thiols, amides, αβ unsaturated carbonyl compounds, etc. in the linker or tail group of an amphipathic molecule. The cross-linking groups irreversibly cross-link, or polymerize, when exposed to ultaviolet light or other radical, anionic or cationic, initiating species. The cross-linking functional groups may be located at specific positions on the hydrophobic portion of an amphipathic molecule. As used herein, the term “diacetylene monomers” refers to single copies of hydrocarbons containing two alkyne linkages (i.e., carbon/carbon triple bonds). In particularly preferred embodiments, the diacetylenes are selected from the group consisting of 10,12-pentacosadiynoic acid, 5,7-pentacosadiynoic acid, sulfate-derivatized 10,12-pentacosadiynoic acid, sulfate-derivatized 5,7-pentacosadiynoic acid, and combinations thereof.

Amphipathic molecules comprise a hydrophilic head group, which may be a chemically reactive head group; a linker or covalent bond between the head and tail groups; and a hydrophobic tail group for self-assembly into films. A mixture of molecules may provide different functional groups on the hydrophilic exposed surface. Amphipathic molecules of interest include lipids, which group includes fatty acids, neutral fats such as triacylglycerols, fatty acid esters and soaps, long chain (fatty) alcohols and waxes, sphingoids and other long chain bases, glycolipids, sphingolipids, carotenes, polyprenols, sterols, and the like, as well as terpenes and isoprenoids. For example, molecules such as diacetylene phospholipids may find use.

Ligand containing molecules. The ligand containing molecules (or specific binding molecules) are amphiphilic molecules having a ligand or specific binding moiety, and hydrophilic head group and a hydrophobic tail group, where the hydrophobic group and hydrophilic group are joined by a covalent bond, or by a variable length linker group. The linker portion may be a bifunctional aliphatic compounds which can include heteroatoms or bifunctional aromatic compounds. Preferred linker portions include, e.g. variable length polyethylene glycol, polypropylene glycol, polyglycine, bifunctional aliphatic compounds, for example amino caproic acid, or bifunctional aromatic compounds.

Usually the ligand or specific binding moiety is covalently or non-covalently bound to the hydrophilic head group. Head groups useful for binding include, for example, biotin, amines, cyano, carboxylic acids, isothiocyanates, thiols, disulfides, α-halocarbonyl compounds, α,β-unsaturated carbonyl compounds, alkyl hydrazines, etc. The amphipathic molecule provides a component of the film, and the ligand or specific binding moiety resides on the surface of the film, where it is accessible for interaction.

Chemical groups that find use in linking a ligand or specific binding moiety to an amphipathic molecule also include carbamate; amide (amine plus carboxylic acid); ester (alcohol plus carboxylic aid), thioether (haloalkane plus sulfhydryl; maleimide plus sulfhydryl); Schiff'base (amine plus aldehyde), urea (amine plus isocyanate), thiourea (amine plus isothiocyanate), sulfonamide (amine plus sulfonyl chloride), disulfide; hyrodrazone, lipids, and the like, as known in the art.

The linkage between ligand or specific binding moiety and amphipathic molecules may comprise spacers, e.g. alkyl spacers, which may be linear or branched, usually linear, and may include one or more unsaturated bonds; usually having from one to about 300 carbon atoms; more usually from about one to 25 carbon atoms; and may be from about three to 12 carbon atoms. Spacers of this type may also comprise heteroatoms or functional groups, including amines, ethers, phosphodiesters, and the like. Specific structures of interest include: (CH₂CH₂O)ₙ where n is from 1 to about 12; (CH₂CH₂NH)ₙ, where n is from 1 to about 12; [(CH₂)ₙ(C═O)NH(CH₂)ₘ]_{z}, where n and m are from 1 to about 6, and z is from 1 to about 10; [(CH₂)ₙOPO₃(CH₂)ₘ]_{z} where n and m are from 1 to about 6, and z is from 1 to about 10. Such linkers may include polyethylene glycol, which may be linear or branched.

The ligand or specific binding moiety may be joined to the amphipathic molecule through a homo- or heterobifunctional linker having a group at one end capable of forming a stable linkage to the hydrophilic head group, and a group at the opposite end capable of forming a stable linkage to the targeting moiety. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidopheny]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio)propionic acid N-hydroxysuccinimide ester (SPDP); N,N′-(1,3-phenylene) bismaleimide; N,N′-ethylene-bis-(iodoacetamide); or 4-(N-maleimidomethyl)cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Other reagents useful for this purpose, include: p,p′-difluoro-m,m′-dinitrodiphenylsulfone (which forms irreversible cross-linkages with amino and, phenolic groups); dimethyl adipimidate (which is specific for amino groups); phenol-1,4-disulfonylchloride (which reacts principally with amino groups); hexamethylenediisocyanate, or diisothiocyanate, or azophenyl-p-diisocyanate (which reacts principally with amino groups); disdiazobenzidine (which reacts primarily with tyrosine and histidine); O-benzotriazolyloxy tetramethuluronium hexafluorophosphate (HATU), dicyclohexyl carbodiimde, bromo-tris (pyrrolidino) phosphonium bromide (PyBroP); N,N-dimethylamino pyridine (DMAP); 4-pyrrolidino pyridine; N-hydroxy benzotriazole; and the like. Homobifunctional cross-linking reagents include bismaleimidohexane (“BMH”).

For example, the specific binding molecules may be formed by converting a commercially available lipid, such as DAGPE, a PEG-PDA amine, DOTAP, etc. into an isocyanate, followed by treatment with triethylene glycol diamine spacer to produce the amine terminated thiocarbamate lipid which by treatment with the paraisothiocyanophenyl glycoside of the targeting moiety produces the desired targeting glycolipids. This synthesis provides a water soluble flexible linker molecule spaced between the amphipathic molecule that is integrated into the film, and the ligand or specific binding moiety that binds to the analyte.

Specific binding moiety. A specific binding moiety, as used herein, refers to all molecules capable of specifically binding to a particular target analyte and forming a bound complex. Thus a ligand and its corresponding target molecule form a specific binding pair.

The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). The complementary members of a specific binding pair are sometimes referred to as a ligand and receptor; or receptor and counter-receptor. For the purposes of the present invention, the two binding members may be known to associate with each other, for example where an assay is directed at detecting compounds that interfere with the association of a known binding pair. Alternatively, candidate compounds suspected of being a binding partner to a compound of interest may be used.

Specific binding pairs of interest include carbohydrates and lectins; peptide ligands and receptor; effector and receptor molecules; hormones and hormone binding protein; enzyme cofactors and enzymes; etc. The specific binding pairs may include analogs, derivatives and fragments of the original specific binding member. For example, a receptor and ligand pair may include peptide fragments, chemically synthesized peptidomimetics, labeled protein, derivatized protein, etc.

Compounds of interest as binding pair members encompass numerous chemical classes, though typically they are organic molecules. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Compounds are found among biomolecules including peptides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of compounds suitable as binding pair members for this invention are those described in The Pharmacological Basis of Therapeutics, Goodman and Gilman, McGraw-Hill, New York, N.Y., (1993) under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Analyte. An analyte is any substance that may be capable of acting as a second specific binding member. Analytes can be proteins, and such are frequently present in a biological sample. Samples can be obtained from the tissues or fluids of an individual, as well as from cell cultures or tissue homogenates. For example, samples can be obtained from whole blood, serum, semen, saliva, tears, urine, fecal material, sweat, buccal, skin, spinal fluid and amniotic fluid. Also included in the term are derivatives and fractions of such cells and fluids. Samples can also be derived from in vitro cell-cultures, including the growth medium, recombinant cells and cell components. The number of cells in a sample will often be at least about 10², usually at least 10³, and may be about 10⁴ or more. The cells may be dissociated, in the case of solid tissues, or tissue sections may be analyzed, and a lysate of the cells prepared.

Array. An array refers to a plurality of agents or ligands, such as a member of a specific binding pair, stably attached to, i.e., immobilized on, a substrate, where the immobilized agents may be spatially located across the surface of the substrate in any of a number of different patterns. A substrate may include one or more arrays, i.e., a plurality of such arrays may be associated with the substrate surface.

### Devices

As summarized above, the present invention includes devices having at least one array for identifying and/or characterizing an analyte, such a peptidic analyte. In general, the subject devices include a substrate, more specifically a hydrophobic substrate, having a polymerized monomolecular film or layer deposited thereon. Associated with the film layer are ligands or members of a specific binding pair. In other words, a plurality of ligands are deposited onto a solid support surface in the form of an “array” or pattern, where the ligands may be the same or different, usually the same. These devices or arrays find use in a variety of applications including, but not limited to, protein identification and characterization, drug discovery, mutation analysis, and the like.

The solid support surface or substrate of the subject invention may be any convenient substrate that is hydrophobic or substantially hydrophobic, has minimal non-specific protein adhesion and generates minimal background signal, including both flexible and rigid substrates. By flexible is meant that the support is capable of being bent, folded or similarly manipulated without breakage. Examples of solid materials that are flexible solid supports with respect to the present invention include membranes, flexible plastic films, and the like. By rigid is meant that the support is solid and does not readily bend, i.e. the support is not flexible. As such, rigid substrates are sufficient to provide physical support and structure to the film deposited thereon. Furthermore, when the rigid supports of the subject invention are bent, they are prone to breakage.

The substrates, may take a variety of configurations ranging from simple to complex. Thug, the substrate could have an overall slide or plate configuration, such as a rectangular, square, oval, elliptical, circular or disc configuration.

The substrates may be fabricated from a variety of materials. In certain embodiments, e.g. where one is interested in the identification and/or characterization of proteins, the materials from which the substrate may be fabricated should ideally exhibit a low level of non-specific binding during complexing (i.e., during the binding or complexing of the binding pair members). For flexible substrates, materials of interest include, but are not limited to, nylon, both modified and unmodified, nitrocellulose, polypropylene, polyester films, such as polyethylene terephthalate, and the like. For rigid substrates, specific materials of interest include: silicon, aluminum oxide surface derivitized with octadecyltriethoxysilane; glass; plastics, e.g. polytetrafluoroethylene, polypropylene, polystyrene, polycarbonate, and blends thereof, and the like; metals, e.g. gold, platinum, and the like; etc., where commercially available ProteinChip H4 from Ciphergen Biosystems, Inc. U.K. is of particular interest.

The substrate surface onto which the fluid composition is deposited may be smooth or substantially planar, or have irregularities, such as depressions or elevations, or have a porous surface, such as is found in porous glass or silica. The surface may be modified with one or more different layers of compounds that serve to modify the properties of the surface in a desirable manner.

As mentioned above, a feature of the subject devices is a layer of a polymerized monomolecular film deposited on at least a portion of the substrate, e.g., a polymerized polydiacetylene monomolecular film. The terms “film” and “coating” herein mean a layer of polymeric material positioned in association with a surface. The term “layer” thus encompasses both “coating” and “film”. In many embodiments of the subject devices, the polymerized monomolecular film is deposited over a substantial area or portion of the substrate surface. By substantial area is meant from about 0.05% to about 100% of the total surface area of the substrate. In certain embodiments, the polymerized monomolecular film is deposited over a relatively small surface area of the substrate surface, e.g., less than about 0.05% of the surface area of the substrate. The layer, and associated ligand as will be described in more detail below, may be present on the surface of the substrate in a continuous layer or may be present as one or more spots on the surface of the substrate.

The spots of polymerized monomolecular film present on the substrate surface may be present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like, where the density of such spots will vary depending on a variety of factors, including, but not limited to, the size of the-particular substrate, the binding pair members of interest, and the like. Each spot may comprise a unique binding pair member, or a plurality of spots may comprise the same binding pair member, e.g. to provide internal controls, duplicate samples, etc.

The size of an individual spot may be at least about 0.1 mm^{2,} about 1 mm², about 3 mm², about 10 mm² or greater. The density of spots on the substrate may be from about 10/cm², about 100/cm², about 1000/cm², about 10,000/cm² or higher.

Regardless of whether the polymerized monomolecular film is present as a continuous layer or as one or more spots on the surface of the substrate, the polymerized monomolecular film is extremely thin. More specifically, the polymerized monomolecular film has about a monomolecular thickness, i.e., the thickness of the layer is only about one molecule thick. However, in certain embodiments of the subject methods, the polymerized diacetylene layer may be thicker than a monomolecular layer.

In certain embodiments, the film includes a high absorption matrix compound or MALDI-MS matrix, which facilitates the use of matrix-associated laser desorption/ionization mass spectroscopy to analyze complexes bound to the device, as will be described in more detail below. A variety of high absorption matrixes are known in the art, including, but not limited to, matrix lipid monomer.

A feature of the subject monomolecular film is that lipids or other amphipathic molecules in the film are is associated or functionalized, i.e., conjugated, with at least one ligand or member of a specific binding pair, also referred to as a probe or receptor to form at least one array on the substrate surface, e.g. arrays of binding agents, such as protein arrays, nucleic acid arrays, small molecule organic ligand arrays, etc. As defined above, by ligand or binding pair member is meant, but is not limited to, a member of a specific binding pair. In certain embodiments, at least one ligand is biotin, integrin antagonists, a specific antibody or specific antigen.

A subject device may include one or a plurality of different ligands or arrays, where each different ligand is capable of binding or capturing a different analyte. For example, a subject device may include a plurality of ligands, such that different ligands may be positioned on different areas of the substrate surface such as spotted on the substrate surface in different regions of the surface, as described above. In one embodiment of the subject devices, at least one ligand is biotin. In yet another embodiment of the subject devices, at least one ligand is an integrin antagonist, e.g. synthetic integrin antagonist.

### Methods of Use

Also provided by the subject invention are methods for identifying and/or characterizing at least one analyte, e.g., protein, carbohydrate, nucleic acid, etc. are provided. The subject methods include analyzing the analyte using mass spectroscopy, e.g., matrix-associated laser desorption/ionization mass spectroscopy (MALDI-MS).

Generally, a sample is contacted with at least one array on the surface of a substrate, such as an array as described above to promote binding of at least one analyte in the sample to an array on the substrate surface. More specifically, a device having a polymerized monomolecular film with at least one functionalized, i.e., conjugated, ligand or member of a specific binding pair, as described above, is provided.

After the provision of the at least one array, a sample is contacted therewith. In other words, a sample suspected of comprising the analyte of interest is contacted with a subject device under conditions sufficient for the analyte to bind to its respective binding pair member that is immobilized on the substrate surface via the diacetylene layer. Thus, if the analyte of interest is present in the sample, it binds to the array at the site of its complementary binding member and a complex is formed on the array surface. The presence of this binding complex on the array surface is then identified.

For example, a sample including streptavidin or the like is first prepared. Following sample preparation, the sample is contacted with at least one array under suitable conditions to promote binding or complexing of the streptavidin with its binding pair complement, i.e., biotin, associated with the film immobilized on the substrate surface, whereby complexes are thus formed between the streptavidin and the biotin attached to the array surface. The at least one array is then typically washed to remove any unbound streptavidin, and any other non-specifically bound molecules from the substrate surface. The bound complexes are then analyzed, for example using mass spectroscopy.

More specifically, mass spectroscopy such as matrix-associated laser desorption/ionization mass spectroscopy (MALDI-MS) is used to identify and characterize analyte, e.g., protein, complexed to the surface of the array. Methods for performing mass spectroscopy are well known in the art (see for example F. Hillenkamp, M. Karas, R. C. Beavis, B. T. Chait, Anal Chem., 1991, 63, 1193). MALDI-MS is particularly well suited for the analysis of proteins, peptides, glycoproteins, oligosaccharides and oligonucleotides. Furthermore, the devices of the subject invention are stable when used with mass spectroscopy and in particular MALDI-MS, i.e., are substantially inert when used in the mass, spectrascope environment, and thus are particularly well suited for use with this mass spectroscopy technique.

Generally, the analyte bound to the surface of the array by its complement binding member, e.g., a protein bound to its complement, is bombarded with a laser light which ionizes the analyte and high absorption matrix, if used. The analyte and matrix ions sputter from the surface of the device, data is then accumulated until a mass to charge (m/z) spectrum of sufficient intensity has been amassed. Ions are separated according to their m/z ratios by measuring the time it takes for ions to travel a flight path through a “field free” region, the heavier ions taking longer than the lighter ions. The ions are detected by a suitable detector such as a photomultiplier tube or the like at the end of the flight path. The ions, and thus the analyte, can thus be identified and characterized by the m/z spectrum generated.

Kits for use in performing analyte identification and/or characterization assays are provided. The subject kits may include one or more subject devices, i.e., a substrate with a polymerized monomolecular film over at least a portion of the substrate, the polymerized monomolecular film having at least one ligand or specific binding pair member associated therewith. Typically, a plurality of subject devices is included. The kits may further include one or more additional components necessary for carrying out an analyte identification and/or characterization assay, such as sample preparation reagents, buffers, labels, and the like. As such, the kits may include one or more containers such as vials or bottles, with each container containing a separate component for the assay, such as reagents for carrying out assays according to the invention. Thus, the kit will comprise in packaged combination, at least one subject array.

The kit may also include a denaturation reagent for denaturing the analyte, hybridization buffers, wash solutions, enzyme substrates, negative and positive controls. Finally, the kits may further include instructions for using the subject devices for performing an assay. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

### EXAMPLES

The following examples are put forth so as to provide those of ordinary skill in the Dart with a complete disclosure and description of how to make and use the present invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The following method describes an exemplary method of manufacturing the subject devices.

### Example 1

Two arrays were prepared according to the subject invention, one using a biotin ligand and another using an integrin antagonist ligand. Accordingly, a mixture of biotin functionalized lipid monomer (0.1-10%) and integrin antagonist (10-30%) was spread in a respective lipid monomer matrix on the surface of water contained in a petri dish. The matrix lipid monomer uniformly disperses the smaller lipid. A UV lamp then irradiated the lipid/water mixture with ultraviolet irradiation at 254 nm for 2 hours to form a conjugated polydiacetylene backbone, seen as a pink floating substance visible to the naked eye, the color indicative of the conjugated polyacteylene backbone. Multiple layers of the conjugated polyacteylene backbone were transferred to H4 ProteinChips available from Ciphergen Biosystems using a horizontal touch technique. The surfaces with the conjugated polyacteylene backbone films were then heated to 165° C. for 1.5 hours.

The films of both arrays were ordered and had good surface coverage, as evidenced by optical fluorescence microscopy. The polymerization of the films affords a red film (λₘₐₓ=540 nm). The films made according to the subject invention formed monolayers with the ethanolamine moieties directed toward the ambient surface and had a monomolecular thickness.

Dose dependant studies were conducted on both the biotin bound films and the integrin antagonist bound films. More specifically, samples including streptavidin at varying concentrations were contacted with the biotin/polyacetylene films and samples including purified integrin αᵥβ₃ at varying concentrations were contacted with the integrin antagonist/polyacetylene films.

The results indicated that the streptavidin and integrin αᵥβ₃ bound to the respective binding members in a dose related manner.

### Example 2

We describe herein a new material for the construction of functionalized thin film-based microarrays and their use in protein capture and subsequent MS detection. This approach, termed Materials for Immobilized Ligand Display (MILD), is based on self-assembled polymerized diacetylene thin films (PDTFs) that are stable to the laser intensities employed in MALDI-MS and display ligands at the interface to capture proteins for MS detection. These materials are simple to construct, exhibit high specificity for different classes of proteins and are amenable to high throughput data acquisition.

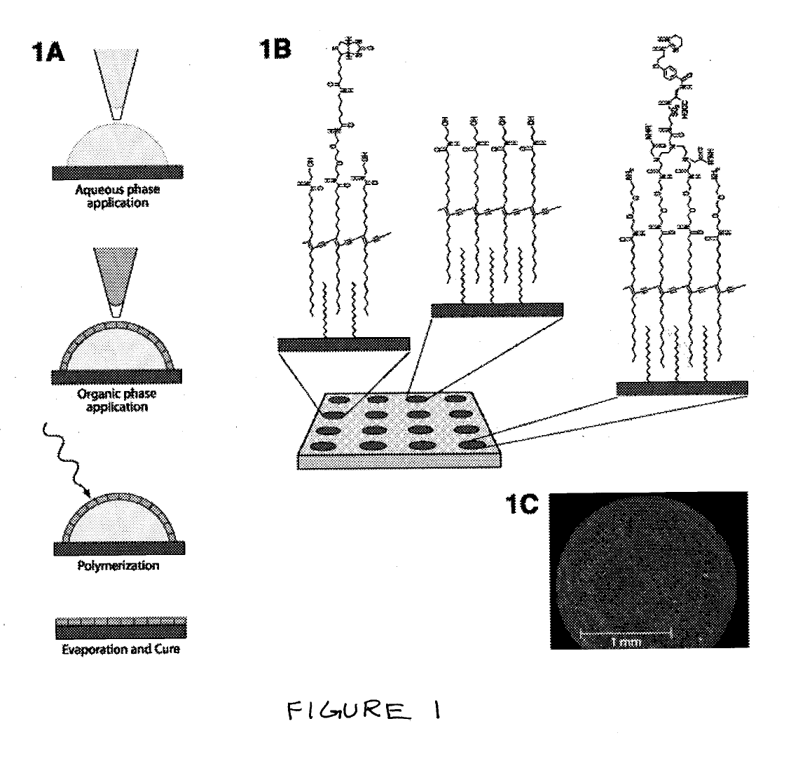

A modified hydrophobic aluminum oxide surface compatible with MALDI-MS or SELDI-MS was used as a platform surface for film deposition. An aqueous subphase was arrayed onto the hydrophobic surface (˜2 mm diameter spot size), and next the polymerizable lipid (dissolved in chloroform) was transferred to the surface of the aqueous subphase (FIG. 1A). Polymerization was achieved by ultraviolet light irradiation (254 nm) followed by curing at room temperature (see Methods). The chemical structure of the PDMFs (with hydroxyl, biotin, and peptidomimetic head group ligands) is shown in FIG. 1B. The PDTFs exhibit extensive surface coverage as demonstrated by the presence of surface fluorescence (FIG. 1C). The XPS profile indicates that the functional head groups are localized at the interface of the PDTFs by the presence of surface nitrogen. The films are stable under standard MALDI-MS operating conditions as no interference from the monomer components of the PDTFs was detected as evidenced by the smooth baseline trace on all mass spectra. Therefore, we have established that stable PDTFs containing ligands can be arrayed on suitable surfaces for MALDI-MS analysis.

To demonstrate the feasibility of MILD-PDTFs in capturing and detecting specific proteins, we used a high affinity ligand-protein binding system (streptavidin-biotin) as a model. The streptavidin-biotin system also allows for future surface modifications with other biotinylated materials, such as antibodies. Streptavidin is a homotetrameric protein that contains four ˜13 kDa biotin binding subunits. The remarkably strong biotin-streptavidin interaction (K_{d} ˜10⁻¹⁵ M) allows for ligand-protein binding as, a model for PDTF-MILD MALDI-MS protein detection approach.

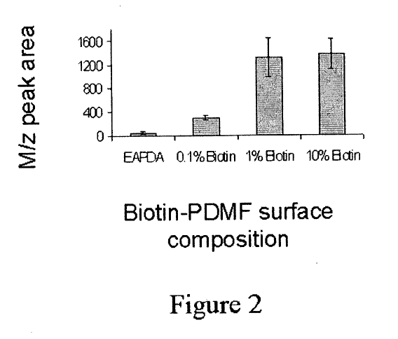

We first constructed the MILD-PDTFs containing 0, 0.1, 1, and 10 mole percent of at biotin functionalized lipid within a matrix of N-(2-hydroxyethyl)-10,12-pentacosadiyanamide (EAPDA), a nonfunctionalized filler. All MILD-PDTF surfaces were incubated with the same amount of streptavidin (5 μl of a 0.55 μM solution, 2.75 pmol total protein). The surfaces were then washed with phosphate buffered saline (PBS) to remove unbound proteins and analyzed by mass spectrometry. The streptavidin monomer (˜13 kDa) was detected on the biotin surface in a ligand concentration dependent manner (FIG. 2). Increasing the biotin concentration from 0.1% to 10% in the PDTFs led to an increased mass/charge (M/z) area in a linear fashion through 1% surface biotin concentration. The best signal intensity was observed on the PDTF containing 10 mole % biotin (or 2.5 pmol/monolayer). As a control, the same amount of streptavidin (2.75 pmol) was incubated on the EAPDA surface containing no biotin, but we were unable to detect significant streptavidin capture on the control surface. The low nonspecific protein binding property of these films is likely due to the preponderance of non-reactive hydroxyl groups of the EAPDA surface. The specific capture of streptavidin demonstrates that the biotin functional head groups are biologically active at the interface of the films and that the ligand is structurally functional after PDTF formation and can be used in protein capture with MALDI-MS analysis.

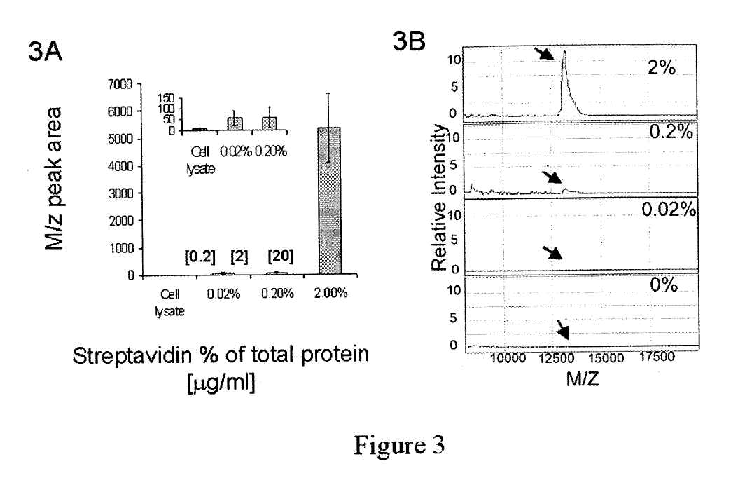

To determine the specificity of protein capture on MILD-PDTFs we investigated streptavidin capture and detection from cellular lysate. Streptavidin was titrated into solutions containing a partially purified cellular lysate (1 mg/ml total protein) from 0.02% to 2% of total protein concentration and the resulting mixtures were applied onto biotin MILD-PDTF surfaces to probe for presence of the protein. Interestingly, streptavidin capture was clearly identified even at a concentration as low as 0.02% (0.2 μg streptavidin/ml) of total protein (FIG. 3A). Even with the complex cellular lysate, there was minimal nonspecific protein adsorption detected on any of the film preparations. Increasing streptavidin capture and detection was related to increasing streptavidin application through 2% (20 μg/ml) of total protein concentration. These results demonstrate that a single protein (streptavidin) can be specifically captured and reproducibly detected from a complex protein solution such as cellular lysate via MILD-MALDI-MS.

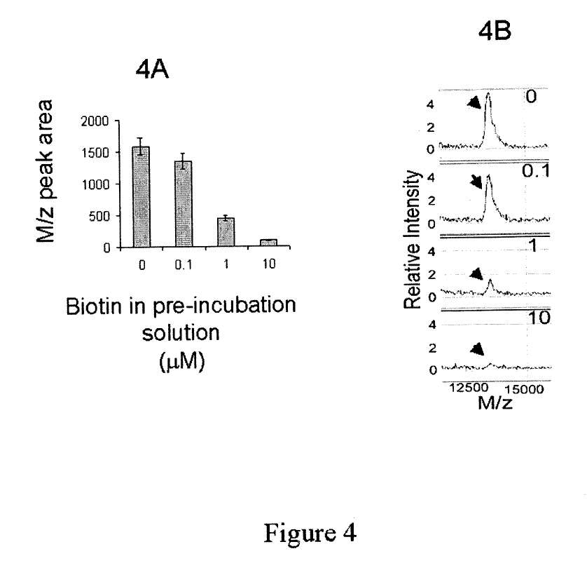

To further demonstrate binding specificity, free biotin was incubated in solution with streptavidin prior to capture with the biotin-PDTF surface in a competitive inhibition experiment. Competition was achieved by increasing the amount of biotin in a constant streptavidin solution from 10 to 1000 mole-percent of the streptavidin. The effect of free biotin competition is clearly seen when the biotin concentration was 100-fold greater than that of streptavidin (FIG. 4) with chip surfaces containing 0.25 pmol/monolayer biotin (1% biotin-PDTF). The remaining free protein (in this case streptavidin) concentration in solution can then be estimated with the MILD-MALDI-MS approach. These findings demonstrate a specific protein-ligand interaction between streptavidin and the immobilized biotin on the PDTF surface.

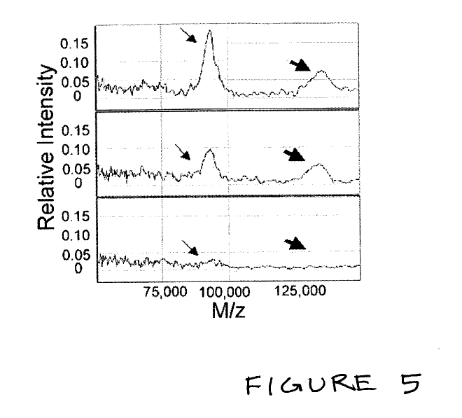

The demonstrated binding specificity of MILD-PDTF using the biotin-streptavidin system encouraged us to extend its application to other biological interactions. Integrins are a family of transmembrane receptors that regulate cell adhesion, growth, and migration. They are also the target of novel anti-cancer drugs in clinical trials. Integrin αᵥβ₃ is a heterodimeric complex of alpha-V (˜130 kDa) and beta-3 (˜95 kDa) subunits. We recently reported on the synthesis of high-avidity integrin αᵥβ₃ antagonist and its application as an integrin-targeting agent in vivo (Hood et al. (2002) Science 296:2404-2407). A functionalized lipid containing this synthetic peptidomimetic was used as a MILD surface. PDTFs containing 30 mole percent functionalized lipid in a matrix of EAPDA were incubated with 200 pmol of purified αᵥβ₃ integrin or αᵥβ₃ solubolized in cellular lysate (1 mg/ml total protein). MILD-MS detected αᵥβ₃ integrin capture in both the purified system and the complex protein mixture (FIG. 5). These results indicate that ligand-receptor interactions can be directly detected even from crude protein mixtures using a customized ligand surface.

The above data demonstrate a material based on polymerized polydiacetylene thin films (PDTFs) that readily displays ligands to capture their binding molecules through molecular recognition that is compatible with MALDI-MS detection systems. The MILD-PDTF technology has a number of advantages that allows it to take the challenging task of high throughput multiplexing analysis: 1) Specificity: our results indicate that proteins binding to ligands coupled to PDTF are highly specific. Washing conditions can be selected to preferentially remove nonspecific protein binding even further. 2) Quantfication: mixing ligand-derived monomers with a monomer matrix prior to polymerization allows even distribution and proper display of the ligand at the material surface. This process also ensures the highly reproducible production of MILD-PDTFs that is essential for quantitative capture and subsequent detection of protein. 3) Versatility: Simple coupling chemistry can generate PDTFs functionalized with a wide variety of ligands. Combined with simple film deposition technology, high-density custom-designed affinity surfaces can be rapidly constructed for substrate binding studies and deposited on a variety of base materials. The PDTFs are a new material that find a use in the developing areas of proteomic analysis.

Materials and Methods

Preparation of monomers. N-(2-hydroxyethyl)-10,12-pentacosadiyanamide (EAPDA) was synthesized as previously described (Wilson & Bednarski (1992) Langmuir 8, 2361-2364). Biotinylated-PEG-PDA was synthesized as previously described (Storrs et al. (1995) Journal of the American Chemical Society 117:7301-7306). The peptidomimetic integrin antagonist was synthesized as previously described (Hood et al., supra.)

Film formation. Monomer mixtures (0.025-2.5 pmol functionalized biotin lipid monomer/layer and integrin antagonist functionalized lipid) were spread on a bead of water covering a hydrophobic surface. The surface area of each spot was 3.14 mm². Ultraviolet irradiation at 254 nm from a UV lamp (Model UVGL-55 Multiband 254/366 nm, UVP Inc, San Gabriel, Calif.) for approximately 30 minutes was used to polymer the films. The surfaces were analyzed under a fluorescent microscope for quality assurance. The surfaces were then cured at room temperature for 1.5 hours.

Cellular lysate preparation. Human M21 melanoma cells were pelleted (approximately 10⁷ cells) and lysed in 500 μl 40 mM Tris (GibcoBRL Life Technologies, Rockville, Md.) pH 8.5 in the presence of protease inhibitor cocktail (Roche, Germany) and sonicated at 4° C. for 30 seconds. The solution was centrifuged at 13,000 rpm for 10 minutes in a microcentrifuge (Centrifuge 5415, Eppendorf, Germany). Total protein concentration in the supernatant was determined by the bicinchonic acid (BCA) assay (Bio-Rad Laboratories, Hercules, Calif.).

Streptavidin and αᵥβ₃ integrin mass spectrometry assay. Five microliters of the following solutions were applied to the polymerized films: streptavidin (Sigma-Aldrich, St. Louis, Mo.) and purified αᵥβ₃ integrin (Chemicon, Temecula, Calif.) diluted in PBS, streptavidin and purified αᵥβ₃ integrin diluted into 1 mg/ml cellular lysate, and streptavidin preincubated with free biotin. All were incubated at high humidity for 30 minutes at room temperature. Following incubation, the solutions were removed from the films and the surfaces were washed three times with PBS and one time with water. The surfaces were allowed to air dry. Saturated sinnapinic acid (SPA, Ciphergen Biosystems, Fremont, Calif.) dissolved in 50% acetonitrile (Sigma-Aldrich, St. Louis, Mo.) containing 0.5% trifluoroacetic acid (Sigma-Aldrich, St. Louis Mo.) was placed to the surface (0.5 μl) and allowed to air dry. The surfaces were then analyzed in a ProteinChip reader (Protein Biology System I or II, Ciphergen Biosystems, Fremont, Calif.).

It is evident from the above description and discussion that the above described invention provides arrays that are easy to use and inexpensive and easy to manufacture. The above described invention provides a number of advantages, including stability to mass spectroscopy analysis and in particular matrix-associated laser desorption/ionization mass spectroscopy (MALDI-MS), customizability, high protein binding specificity and low non-specific protein adhesion. As such, the subject invention represents a significant contribution to the art.

What is claimed is:

1. A device for identifying and characterizing an analyte, said device comprising:
(a) a substrate; and
(b) a polymerized monomolecular film deposited on at least a portion of said substrate surface, wherein said polymerized monomolecular film comprises at least one specific binding moiety.

2. The device according to claim 1, comprising a plurality of spots of said polymerized monomolecular film deposited on at least a portion of said substrate surface, wherein each of said spots comprises a unique specific binding moiety.

3. The device according to claim 1, wherein said monomolecular film comprises a mixture of a cross-linking self-assembling monomers and amphipathic molecules comprising a specific binding moiety.

4. The device according to claim 3, wherein the ratio of said cross-linking self-assembling monomers to said amphipathic molecules comprising a specific binding moiety is at least about 0.1 mole percent.

5. The device according to claim 4, wherein said self-assembling monomers are diacetylene monomers.

6. The device according to claim 5, wherein said diacetylene monomers are 10,12-pentacosadiynoic acid.

7. The device according to claim 1, wherein said specific binding moiety is covalently or non-covalently bound to the hydrophilic head group of an amphipathic molecule.

8. The device according to claim 7, wherein said amphipathic molecule is a lipid.

9. A method of performing an analyte identification and characterization assay, said method comprising:
(a) providing a device, wherein said device comprises:
(i) a substrate; and
(ii) a polymerized monomolecular film deposited on at least a portion of said substrate surface, wherein said polymerized monomolecular film comprises at least one specific binding moiety;
(b) contacting said device with a sample comprising at least one analyte complementary to said at least one specific binding moiety;
(c) forming at least one complex between said at least one analyte and said at least one ligand; and
(d) analyzing said complex using mass spectroscopy techniques.

10. The method according to claim 9, wherein said device comprises a plurality of spots of said polymerized monomolecular film deposited on at least a portion of said substrate surface, wherein each of said spots comprises a unique specific binding moiety.

11. The method according to claim 9, wherein said monomolecular film comprises a mixture of a cross-linking self-assembling monomers and amphipathic molecules comprising a specific binding moiety.

12. The method according to claim 11, wherein the ratio of said cross-linking self-assembling monomers to said amphipathic molecules comprising a specific binding moiety is at least about 0.1 mole percent.

13. The method according to claim 12, wherein said self-assembling monomers are diacetylene monomers.

14. The method according to claim 13, wherein said diacetylene monomers are 10,12-pentacosadiynoic acid.

15. The method according to claim 11, wherein said specific binding moiety is covalently or non-covalently bound to the hydrophilic head group of an amphipathic molecule.

16. The method according to claim 15, wherein said amphipathic molecule is a lipid.
